(19) United States
(12) Patent Application Publication
(10) Pub. No.: US 2006/0073572 A1
(43) Pub. Date: Apr. 6, 2006

(54) Cytokine structurally related to IL-17

(75) Inventors: Shau-Ku Huang, Towson, MD (US); Gregory Germino, Chevy Chase, MD (US); David M. Essayan, Oak Park, CA (US); Luiz F. Onuchic, Sao Paulo (BR); Mio Kawaguchi, Columbia (JP); Xiao-Dong Li, Baltimore, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD (US)

(21) Appl. No.: 11/024,980

(22) Filed: Dec. 30, 2004

Related U.S. Application Data
(63) Continuation of application No. 10/467,368, now abandoned.
(60) Provisional application No. 60/267,676, filed on Feb. 9, 2001.
(60) Provisional application No. 60/287,633, filed on Apr. 30, 2001.

(51) Int. Cl. C07K 14/54; C07H 21/04; C12P 21/02

(52) U.S. Cl. 435/69.5; 435/320.1; 435/325; 530/351; 536/23.5

(57) ABSTRACT

The cytokine ML-1 is a target for treating airway inflammation. Candidate therapeutic agents for treating airway inflammation can be screened for the ability to bind to an ML-1 protein or polynucleotide, from the ability to decrease functional properties of ML-1, or for the ability to decrease ML-1 gene expression. Pharmaceutical compositions comprising such reagents can be used to treat airway inflammation associated with a variety of lung disorders, such as asthma, chronic obstructive pulmonary disease, emphysema, allergic inflammatory responses, and cystic fibrosis.

This application claims priority to and incorporates by reference co-pending provisional applications Ser. No. 60/267,676 filed Feb. 9, 2001 and Ser. No. 60/287,633 filed Apr. 30, 2001.

This invention resulted from research funded in whole or in part by National Institutes of Health Grant No. AI-34002. The Federal Government has certain rights in this invention.

### FIELD OF THE INVENTION

The invention relates to a novel cytokine and its role in airway inflammatory responses.

### BACKGROUND OF THE INVENTION

Cytokines are small, secreted proteins which bind to receptors on cell membranes and which regulate many important cellular processes, such as cell growth, differentiation, and response to various stimuli. For example, epithelial and endothelial cells play an important role in the regulation of inflammatory process via their abilities to express a wide range of cytokines, such as IL-6 and IL-8 (21-24).

Cytokines can be divided into families of related proteins. The amino acid sequences of member cytokines in different subfamilies usually do not have significant homologies, due to their diversity of the functions. U.S. Pat. No. 6,245,550. Recent cloning and sequencing studies have demonstrated that there is a family of IL-17-related genes with potential proinflammatory functions. Members of the IL-17 gene family are classified based on amino acid sequence similarity. However, the genes are selectively expressed in different tissues and are dispersed in the genome (1, 14, 15). Distinct function among members of the IL-17 gene family has been demonstrated (2, 16, 25-27).

Human IL-17 is a T-cell derived, homodimeric protein which exhibits pleiotropic biological activities (1-3). Whereas the expression of IL-17 is restricted to activated T cells, the IL-17 receptor is widely expressed. IL-17 stimulates the production of IL-6, IL-8, granulocyte/macrophage colony-stimulating factor (GM-CSF), stem cell factor, and prostaglandin E₂ from various cell types, such as fibroblasts, keratinocytes, and renal and airway epithelial cells (3-7). In addition, elevated IL-17 mRNA expression has been found in mononuclear cells from patients with multiple sclerosis (8), in patients with rheumatoid arthritis (9), and in patients with systemic lupus erythematosus (10), which suggests a role for IL-17 in the initiation or maintenance of inflammatory responses. In the airway, IL-17 induces expression of the C—X—C chemokines, IL-8, and macrophage inflammatory protein-2 (MIP-2), which selectively recruit neutrophils into the airway (11). IL-17 also acts synergistically with IFN-γ to induce ICAM-1 expression on epithelial cells (12); this induction is associated with airway inflammation seen in bronchial asthma (13).

Two members of the IL-17 gene family, IL-17B and IL-17C, each share approximately 27% amino acid identity with IL-17 (14, 15). IL-17B mRNA is expressed in adult pancreas, small intestine, and stomach, whereas IL-17C mRNA is not detected in the same set of adult tissues. Interestingly, neither IL-17B nor IL-17C is detected in activated CD4+ T cells (14, 15). Both IL-17B and IL-17C stimulate the release of tumor necrosis factor and IL-1 from a monocytic cell line, THP-1. However, IL-17B and IL-17C are not able to stimulate IL-6 production from human fibroblasts and do not bind to the human IL-17 receptor. A newly discovered member of the IL-17 gene family, IL-17E, induces activation of NF-kB and IL-8 via a distinct receptor (16).

Taken together, these data indicate that members of the family of IL-17-related cytokines differ in patterns of expression and pro-inflammatory responses. Because of the importance of cytokines in various disease processes, there is a need in the art to identify related molecules in this cytokine family.

### BRIEF SUMMARY OF THE INVENTION

It is an object of the invention to provide reagents and methods for treating airway inflammation, as well as methods of screening for candidate therapeutic agents for treating airway inflammation. These and other objects of the invention are provided by one or more of the embodiments described below.

One embodiment of the invention is an isolated and purified human ML-1 protein comprising a first polypeptide segment comprising the amino acid sequence shown in SEQ ED NO:2.

Another embodiment of the invention is an isolated and purified human ML-1 protein comprising an amino acid sequence which differs from the amino acid sequence shown in SEQ ID NO:2 by between one and ten conservative amino acid substitutions and which (a) induces expression of IL-6, IL-8, and ICAM-1 in primary bronchial epithelial cells, (b) induces ERK1/2 activity in human primary bronchial epithelial cells and in human umbilical vein endothelial cells, (c) increases neutrophil chemotaxis, and (d) is expressed in activated CD4+ T cells, basophils, peripheral blood monocytes, and mast cells.

Yet another embodiment of the invention is an isolated and purified polypeptide comprising a first polypeptide segment which comprises between 10 and 108 contiguous amino acids of a human ML-1 protein as shown in SEQ ID NO:2.

Still another embodiment of the invention is a purified preparation of antibodies which specifically bind to a human ML-1 protein comprising the amino acid sequence of SEQ ID NO:2.

Even another embodiment of the invention is an isolated and purified polynucleotide which encodes a human ML-1 protein comprising the amino acid sequence shown in SEQ ID NO:2.

A further embodiment of the invention is a cDNA molecule which encodes a human ML-1 protein comprising the amino acid sequence shown in SEQ ID NO:2.

Another embodiment of the invention is an isolated and purified single-stranded probe comprising between 12 and 329 contiguous nucleotides of a coding sequence for a human ML-1 protein or the complement thereof. The ML-1 protein comprises the amino acid sequence shown in SEQ ID NO:2.

Still another embodiment of the invention is an isolated and purified antisense oligonucleotide comprising a first sequence of between 12 and 330 contiguous nucleotides which is complementary to a second sequence of between 12 and 330 contiguous nucleotides found in a coding sequence for a human ML-1 protein which comprises the amino acid sequence shown in SEQ ID NO:2.

Even another embodiment of the invention is a container comprising a set of primers. The set comprises a first primer comprising at least 12 contiguous nucleotides which is complementary to a contiguous sequence of nucleotides located at the 5′ end of the coding strand of a double-stranded polynucleotide which encodes a human ML-1 protein as shown in SEQ ID NO:2 and a second primer comprising at least 12 contiguous nucleotides which is complementary to a contiguous sequence of nucleotides located at the 5′ end of the non-coding strand of the polynucleotide.

Yet another embodiment of the invention is an expression construct, which comprises a coding sequence for a human ML-1 protein comprising the amino acid sequence shown in SEQ ID NO:2 and a promoter which is located upstream from the coding sequence and which controls expression of the coding sequence.

A further embodiment of the invention is a host cell comprising an expression construct. The expression construct comprises a coding sequence for a human ML-1 protein comprising the amino acid sequence shown in SEQ ID NO:2 and a promoter which is located upstream from the coding sequence and which controls expression of the coding sequence.

Another embodiment of the invention is a method of producing a human ML-1 protein. A host cell is cultured in a culture medium. The host cell comprises an expression construct comprising (a) a coding sequence for a human ML-1 protein comprising the amino acid sequence shown in SEQ ID NO:2 and (b) a promoter which is located upstream from the coding sequence and which controls expression of the coding sequence. The step of culturing is carried out under conditions whereby the protein is expressed. The protein is recovered from the culture medium.

Still another embodiment of the invention is a method of detecting an ML-1 expression product. A test sample is contacted with a reagent that specifically binds to an expression product of the ML-1 coding sequence. The test sample is assayed to detect binding between the reagent and the expression product. The test sample is identified as containing an ML-1 expression product if binding between the reagent and the expression product is detected.

Yet another embodiment of the invention is a method of increasing expression in a human cell of interleukin-6 (IL-6) or interleukin-8 (IL-8). A human cell which is capable of expressing IL-6 or IL-8 is provided with a human ML-1 protein comprising the amino acid sequence shown in SEQ ID NO:2. Expression of IL-6 or IL-8 is thereby increased in the cell relative to expression in the cell in the absence of the ML-1 protein.

A further embodiment of the invention is a method of increasing expression of ICAM-1 in a human endothelial cell. An endothelial cell is provided with a human ML-1 protein comprising the amino acid sequence shown in SEQ ID NO:2. Expression of the ICAM-1 is thereby increased in the cell relative to expression in the cell of ICAM-1 in the absence of the ML-1 protein.

Another embodiment of the invention is a method of treating. An effective amount of a reagent that either (a) decreases expression of a human ML-1 gene that encodes a human ML-1 protein comprising the amino acid sequence shown in SEQ ID NO:2 or (b) decreases effective levels of the ML-1 protein is administered to a patient with airway inflammation. Symptoms of the airway inflammation are thereby reduced.

Still another embodiment of the invention is a method of inhibiting human neutrophil chemotaxis. A human neutrophil is contacted with an effective amount of a reagent that either (a) decreases expression of a human ML-1 gene which encodes an ML-1 protein comprising the amino acid sequence shown in SEQ ID NO:2 or (b) decreases effective levels of the ML-1 protein. Chemotaxis of the neutrophil is thereby inhibited relative to chemotaxis of the neutrophil in the absence of the reagent.

Yet another embodiment of the invention is a method of screening for candidate therapeutic agents that may be useful for treating airway inflammation. A human ML-1 protein comprising the amino acid sequence shown in SEQ ID NO:2 is contacted with a test compound. Binding between the ML-1 protein and the test compound is assayed. A test compound that binds to the ML-1 protein is identified as a candidate therapeutic agent that may be useful for treating airway inflammation.

A further embodiment of the invention is a method of screening for candidate therapeutic agents that may be useful for treating airway inflammation. Expression of a polynucleotide encoding a human ML-1 protein comprising the amino acid sequence of SEQ ID NO:2 is assayed in the presence and absence of a test compound. A test compound that decreases the expression is identified as a candidate therapeutic agent that may be useful for treating airway inflammation.

Another embodiment of the invention is a pharmaceutical composition comprising a reagent which binds to an expression product of a human ML-1 gene which encodes an ML-1 protein comprising the amino acid sequence shown in SEQ ID NO:2 and a pharmaceutically acceptable carrier.

Yet another embodiment of the invention is a pharmaceutical composition comprising a human ML-1 protein comprising the amino acid sequence shown in SEQ ID NO:2 and a pharmaceutically acceptable carrier.

Still another embodiment of the invention is a pharmaceutical composition comprising a polynucleotide encoding a human ML-1 protein comprising the amino acid sequence shown in SEQ ID NO:2 and a pharmaceutically acceptable carrier.

The invention thus provides reagents and methods for treating airway inflammation, as well as methods of screening for candidate therapeutic agents for treating airway inflammation.

### BRIEF DESCRIPTION OF THE DRAWINGS

FIG. 1. Amino acid sequence alignment of ML-1 (SEQ ID NO:2), IL-17 (SEQ ID NO:3), IL-17C (SEQ ID NO:4), and IL-17B (SEQ ID NO:5). The residues in each protein which are identical to those of the ML-1 sequence are bold. Conserved cysteine residues are indicated by arrows.

FIGS. 2A and 2B. Expression patterns of ML-1 gene. FIG. 2A, RT-PCR analysis of ML-1 expression in different cell types. Lane 1, activated basophils; lane 2, activated peripheral blood monocytes (PBMCs); lane 3, activated Th2 clone; lane 4, activated Th1 clone; lane 5, activated Th0 clone. FIG. 2B, Expression of ML-1 gene at sites of airway inflammation. Detection of gene expression in BAL cells from four asthmatic patients (BAL#638, #646, #688 and #1081) challenged with allergen (Ag) and saline control (NS). Amplification of G3PDH was performed as a positive control.

FIGS. 3A-3C. FIG. 3A, Expression of ML-1 protein in COS-7 cells. COS-7 cells were transfected with a gene construct containing full-length ML-1 sequence and a tag sequence encoding poly (His) peptide. ML-1-His fusion proteins were purified from supernatants of the transfected cells using a His affinity column, run on an 8-16% SDS-polyacrylamide gel under reducing conditions, and analyzed by Western Blot using anti-His monoclonal antibody. Lane 1 represents a mock-transfected sample. Lane 2 represents an ML-1-transfected sample. The location of a 19.5 kd marker is indicated. FIG. 3B, Effect of ML-1 on IL-8 gene expression. Human primary bronchial epithelial cells (NHBE cells) were treated with different stimuli. The cells were harvested 4 hours after stimulation, and total RNA was extracted and subjected to RT-PCR using IL-8 and G3PDH primers. Lane 1, medium only; lane 2, cells treated with rIL-17 (100 ng/ml); lane 3, cells treated with purified His-tagged protein (Positope, 100 ng/ml); lane 4, cells treated with ML-1 (100 ng/ml). FIG. 3C, Effect of ML-1 on IL-8 protein production. NHBE cells were treated with the same stimuli used in FIG. 3B, and the supernatants were harvested 48 hours after stimulation.

FIG. 4. Effect of ML-1 on ICAM-1 surface expression on NHBE cells by Flow Cytometry. NHBE cells were treated for 48 hours under following conditions: medium only, rIL-17 (100 ng/ml), purified His-tagged protein (Positope, 100 ng/ml), or ML-1 (100 ng/ml) as indicated. The mean fluorescence intensity of ICAM-1 surface expression was measured. The experiment was conducted four times. The results shown are from a representative experiment. * p<0.01.

FIG. 5. Analysis of IL-6 (FIG. 5A) and IL-8 (FIG. 5B) expression in primary bronchial epithelial cells (PBECs) and human umbilical vein endothelial cells (HUVECs) stimulated by ML-1. The IL-6 and IL-8 protein release in the supernatant was determined by ELISA as described in Example 8. The results were expressed as mean+SD (n=6). *p<0.05 was considered significant vs control.

FIGS. 6A-6D. Kinetic activation of ERK1/2 by ML-1 in PBECs (FIG. 6A) and HUVECs (FIG. 6B). The cells were incubated with or without ML-1 (100 ng/ml) for different time points as indicated. Western blotting analysis was performed by using antibodies against different MAP kinases as described in Example 8. FIGS. 6C and 6D. Effect of PD98059 on ML-1-induced phosphorylation of ERK1/2 in PBECs (FIG. 6C) and HUVECs (FIG. 6D). The cells were preincubated with PD98059 (10 mM) or DMSO vehicle control for 1 hour, followed by stimulation of PBECs and HUVECs with medium or ML-1 for 20 minutes or 10 minutes, respectively. The results shown are representative of three separate experiments.

FIG. 7A-7D. Effect of PD98059 on IL-6 (FIG. 7A) and IL-8 (FIG. 7B) protein production in PBECs and HUVECs. The cells were preincubated with varying concentrations of PD98059, SB202190, or DMSO vehicle control for 1 hour, followed by stimulation with ML-1 (100 ng/ml) for 24 hours. The results are expressed as the mean+SD (n=6). *P<0.05 when compared to cultures without the addition of PD98059. FIGS. 7C and 7D. Semi-quantitative analysis of gene expression for IL-6 (FIG. 7C) and IL-8 (FIG. 7D) by RT-PCR. The cells were preincubated as described above and treated with ML-1 for 4 hours. RT-PCR was performed as described in Example 8. The results shown are representative of three separate experiments.

FIGS. 8A-8B. Expression patterns of ML-1 gene. FIG. 8A, RT-PCR analysis of ML-1 expression in different cell types. Lane 1, activated basophils; lane 2, activated PBMCs; lane 3, activated Th2 clone; lane 4, activated TH1 clone; lane 5, activated Th0 clone. FIG. 8B, expression of ML-1 gene at sites of airway inflammation. Detection of gene expression in BAL cells from four asthmatic patients (BAL#638, #646, #688, and #1081) challenged with allergen (Ag) and saline control (NS). Amplification of G3PDH was performed as a positive control.

### DETAILED DESCRIPTION OF THE INVENTION

One aspect of the present invention is a new member of the IL-17 cytokine family, ML-1, which may play a role in proinflammatory responses. The ML-1 gene is located on the same genomic DNA clone (Accession #AL391221) as the IL-17 gene, which is located about 50 kb telemeric to the ML1 gene. The genes are in a tail-to-tail orientation, suggesting a potential gene-duplication event. ML-1 is expressed in liver, lung, ovary, and fetal liver. In contrast, IL-17 expression is restricted to activated peripheral blood monocytes (PBMCs) and activated Th0 cells. ML-1 also is expressed in activated CD4+ T cells, basophils, PBMCs, and mast cells (27). ML-1 shares a significant degree of sequence homology with human IL-17 (2, 16, 25-27).

ML-1 induces expression of IL-6, IL-8, and intercellular adhesion molecule (ICAM)-1 in primary bronchial epithelial cells. Expression of ML-1 itself is significantly upregulated in bronchoalveolar lavage cells of asthmatic subjects following exposure to segmental allergen (27).

ML-1-induced expression of IL-6 and IL-8 is mediated through the activation of ERK1/2 in both primary bronchial epithelial cells (PBECs) and human umbilical vein endothelial cells (HUVECs). Interestingly, previous data showed that ERK1/2, but not p38 or JNK, may play an important role in cytokine release in primary epithelial cells (28), although all three members of MAP kinase family are involved in cytokine expression. Indeed, IL-17, which shows high homology to ML-1, also activates only ERK1/2 kinase in PBECs (34). It is noted, however, that induction of both IL-6 and IL-8 is not completely inhibited by ERK1/2 kinase inhibitor, PD98059, suggesting that an additional signaling pathway is involved in the induction of these cytokines by ML-1 in these cell types.

ML1 Proteins

An isolated and purified ML-1 protein is separated from other compounds that normally associate with the ML-1 protein in a cell in which it is synthesized, such as other proteins, carbohydrates, or lipids. Isolated and purified ML-1 proteins are in preparations that are free or at least 70, 80, or 90% free of other protein molecules. As used herein, the term “ML-1 protein” includes full-length, naturally occurring ML-1 protein comprising the amino acid sequence shown in SEQ ID NO:2, as well as polypeptide fragments of that protein, fusion proteins comprising all or a portion of SEQ ID NO:2, and naturally or non-naturally occurring variants of full-length ML-1, ML-1 polypeptide fragments, and ML-1 fusion proteins which retain the biological activities of full-length, naturally occurring ML-1.

Polypeptide Fragments and ML-1 Variants

Polypeptide fragments of human ML-1 protein comprise between 10 and 108 contiguous amino acids of SEQ ID NO:2 (for example, 10, 15, 20, 25, 30, 50, 75, or 100 contiguous amino acids). Naturally or non-naturally occurring variants of full-length human ML-1 protein or polypeptide fragments thereof retain the biological activities of native ML-1 protein, including the ability to induce IL-6, IL8, or ICAM-1 expression in primary bronchial epithelial cells, to induce ERK1/2 activity in primary bronchial epithelial cells or in human umbilical vein endothelial cells, to increase neutrophil chemotaxis, and to be over-expressed in activated CD4+ T cells, basophils, peripheral blood monocytes, and mast cells. “Induction of expression” means any statistically significant increase in expression over a baseline level of expression measured in the absence of ML-1, e.g., a 10, 20, 25, 50, 75, 80, or 90% increase. “Induction of ERK 1/2 activity” means any statistically significant increase in activity over a baseline level of activity measured in the absence of ML-1, e.g., a 10, 20, 25, 50, 75, 80, or 90% increase. Baseline expression or activity can be undetectable or can simply be expression or activity at a lower level than that in the presence of ML-1. Similarly, “increased neutrophil chemotaxis” means any statistically significant increase in speed of neutrophil movement and/or an increase in the percentage of a populations of neutrophils exhibiting chemotaxis when measured with respect to a baseline speed or percentage determined in the absence of ML-1.

ML-1 variants preferably have amino acid sequences which are at least about 85, 90, 95, 96, 97, 98, or 99% identical to the amino acid sequence shown in SEQ ID NO: 2 or a fragment thereof. Percent identity between a putative human ML-1 polypeptide variant and all or the corresponding portion of the amino acid sequence of SEQ ID NO:2 can be determined by conventional methods, such as BLAST or FASTA. See, for example, Altschul et al., Bull. Math. Bio. 48:603, 1986, Henikoff & Henikoff, Proc. Natl. Acad. Sci. USA 89:10915, 1992, Pearson & Lipman, Proc. Nat'l Acad. Sci. USA 85:2444, 1988, and Pearson, Meth. Enzymol. 183:63, 1990.

Variations in percent identity can be due, for example, to amino acid substitutions, insertions, or deletions. “Amino acid substitutions” are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine. Preferred variants differ from the amino acid sequence shown in SEQ ID NO:2 by between 1 and 10 conservative amino acid substitutions (i.e., 1, 2, 3, 4, 5, 6, 7, 8, 9, or 10 conservative amino acid substitutions).

The term “ML-1 protein” also includes full-length ML-1 protein or fragments thereof that are modified during or after translation, as well as chemically modified derivatives that may provide additional advantages such as increased solubility, stability and circulating time of the protein, or decreased immunogenicity. See U.S. Pat. No. 4,179,337.

Whether an amino acid change or other modification results in a biologically active ML-1 variant can readily be determined by assaying for functional properties of ML-1 described above. Assays for these functions are described in the Examples, below.

Fusion Proteins

Fusion proteins are useful for purifying ML-1, for generating antibodies against ML-1, and for use in various assay systems. For example, fusion proteins can be used to identify proteins that interact with portions of human ML-1. Protein affinity chromatography or library-based assays for protein-protein interactions, such as the yeast two-hybrid or phage display systems, can be used for this purpose. Such methods can also be used as drug screens.

Human ML-1 fusion proteins comprise two polypeptide segments joined together by means of a peptide bond. The first polypeptide segment comprises a sequence of between 10 and 109 contiguous amino acids (e.g., 10, 15, 20, 25, 30, 50, 75, 100, or 109) of SEQ ID NO:2 or of a biologically active variant, such as those described above. The second polypeptide segment can be a full-length protein or a protein fragment but is not an ML-1 protein or fragment thereof. Proteins such as β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT) are suitable for use as the second polypeptide segment. The second polypeptide segment also can be an epitope tag including, but not limited to, a histidine (His) tag, a FLAG tag, an influenza hemagglutinin (HA) tag, a Myc tag, an S-tag, a VSV-G tag, or a thioredoxin (Trx) tag. Other suitable second polypeptide segments include maltose binding protein (MBP), Lex A DNA binding domain (DBD), GAL4 DNA binding domain, and herpes simplex virus (HSV) basepair16 protein. If desired, a coding sequence for a fusion protein can be engineered to contain a cleavage site located between the sequence encoding ML-1 and the nucleotide sequence encoding the second polypeptide segment, so that the ML-1 sequence can be cleaved and purified away from the second polypeptide segment.

ML-1 Polynucleotides

The invention provides polynucleotide molecules that encode the ML-1 proteins described above. Human ML-1 polynucleotides can be single- or double-stranded and comprise at least a portion of a coding sequence or the complement of a coding sequence for an ML-1 protein. Human ML-1 polynucleotides include naturally occurring coding sequences for human ML-1 protein, particularly the nucleotide sequence shown in SEQ ID NO:1, as well as degenerate versions thereof that encode SEQ ID NO:2. The invention also provides complementary DNA (cDNA) molecules (see GenBank Accession No. AF332389), polynucleotides that encode biologically active variants of ML-1 protein, and polynucleotide fragments comprising between 12 and 329 contiguous nucleotides of SEQ ID NO: 1 or its complement (for example, 12, 15, 20, 21, 25, 50, 75, 100, 125, 150, 175, 200, 225, 250, 275, 300, 325, or 329 contiguous nucleotides). These fragments can be used, for example, as hybridization probes, as primers, or as antisense oligonucleotides. Sets of polynucleotides to be used as primers for amplifying ML-1 polynucleotides include one primer which hybridizes to a sequence located at the 5′ end of the coding strand of a double-stranded polynucleotide to be amplified (ie., 5′ of the sequence to be amplified) and a second primer which is complementary to a contiguous sequence of nucleotides located at the corresponding 5′ end of the non-coding strand of the polynucleotide.

An isolated and purified human ML-1 polynucleotide is isolated free of other cellular components such as membrane components, other polynucleotides, proteins, and lipids. Isolated and purified polynucleotides are in preparations that are free or at least about 70, 80, 90, 95, 98, or 99% free of other molecules.

Preparation of ML-1 Polynucleotides and Proteins

Polynucleotides

Polynucleotides can be made by a cell and isolated using standard nucleic acid purification techniques. Methods for isolating polynucleotides are routine and are known in the art. Any such technique for obtaining a polynucleotide can be used to obtain ML-1 polynucleotides. For example, restriction enzymes and probes can be used to isolate polynucleotide fragments that comprise ML-1 nucleotide sequences.

Alternatively, synthetic chemistry techniques can be used to synthesize ML-1 polynucleotides. The degeneracy of the genetic code allows alternate nucleotide sequences which will encode a human ML-1 protein to be synthesized. Polynucleotides also can be synthesized using specific primers and an amplification technique, such as polymerase chain reaction (PCR).

Human ML-1 cDNA molecules can be made with standard molecular biology techniques, using ML-1 mRNA as a template. Human ML-1 cDNA molecules can thereafter be replicated using molecular biology techniques known in the art and disclosed in manuals such as Sambrook et al. (1989). For example, an amplification technique, such as PCR, can be used to obtain additional copies, using either human genomic DNA or cDNA as a template.

Proteins

Human ML-1 protein can be purified from any human cell which naturally expresses the protein, including lung, ovary, and fetal or adult liver, as well as activated CD4+ T cells, basophils, PMBCs, and mast cells. Methods well-known in the art, including, but not limited to, size exclusion chromatography, ammonium sulfate fractionation, ion exchange chromatography, affinity chromatography, and preparative gel electrophoresis, can be used to purify ML-1 proteins. Typically, ML-1-expressing cells will be cultured, and secreted ML-1 protein will be purified from the culture medium.

ML-1 proteins also can be synthesized chemically, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of ML-1 proteins can be separately synthesized and combined using chemical methods to produce a full-length molecule.

Preparation of Recombinant Proteins

Preferably, recombinant DNA methods are used to prepare ML-1 proteins. The invention provides expression constructs for this purpose. Expression constructs of the invention typically contain a coding sequence for an ML-1 protein, as well as the necessary elements for the transcription and translation of the coding sequence. Expression of the coding sequence is under the control of a promoter which is located 5′ (upstream) of the coding sequence. Depending on the host cell which is used, any number of suitable transcription and translation elements, including constitutive and inducible promoters, can be used. For example, in bacterial systems, inducible promoters such as the hybrid lacZ promoter of the BLUESCRIPT phagemid (Stratagene, LaJolla, Calif.) or pSPORT1 plasmid (Life Technologies) can be used. The baculovirus polyhedrin promoter can be used in insect cells. Promoters or enhancers derived from the genomes of plant cells (e.g., heat shock, RUBISCO, and storage genes) or from plant viruses (e.g., viral promoters or leader sequences) can be cloned into an expression construct. In mammalian cell systems, promoters from mammalian genes are preferred.

Methods which are well-known to those skilled in the art can be used to build expression constructs containing sequences encoding ML-1 polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

Mechanical methods, such as microinjection, liposome-mediated transfection, electroporation, or calcium phosphate precipitation, can be used to introduce an ML-1 expression construct into a host cell. Alternatively, expression constructs of the invention can be incorporated into polynucleotide delivery vehicles. Polynucleotide delivery vehicles can be, for example, a plasmid, a viral-based vector, or an ML-1 polynucleotide in conjunction with a liposome or a condensing agent. Numerous polynucleotide delivery vehicles are known in the art and can be used to deliver ML-1 polynucleotides to a host cell.

A host cell strain can be chosen for its ability to modulate the expression of ML-1 protein or to process the ML-1 protein in a desired fashion. Such modifications of the protein may include acetylation, carboxylation, glycosylation, phosphorylation, lipidation, and acylation. Different host cells that have specific cellular machinery and characteristic mechanisms for post-translational activities (e.g., CHO, HeLa, MDCK, HEK293, and W138) are available from the American Type Culture Collection (ATCC; 10801 University Boulevard, Manassas, Va. 20110-2209) and can be chosen to ensure the correct modification and processing of a foreign protein. Host cells can be prokaryotic or eukaryotic. For example, bacterial, yeast, plant, insect, and mammalian, including human, cells can be used to produce recombinant ML-1 proteins. ML-1-producing host cells can be cultured using standard cell culture techniques, and secreted ML-1 protein can then be purified from the culture medium.

Antibodies

Any type of antibody known in the art can be generated to bind specifically to an epitope of a human ML-1 protein. “Antibody” as used herein includes intact immunoglobulin molecules, as well as fragments, such as Fab, F(ab′)2, and Fv fragments, which are capable of binding an epitope of a human ML-1 protein. Typically, at least 6, 8, 10, or 12 contiguous amino acids are required to form an epitope. However, epitopes which involve non-contiguous amino acids may require more, e.g., at least 15, 25, or 50 amino acids. Purified antibody preparations of the invention are those in which a majority of the antibodies present in the preparation specifically bind to human ML-1.

An antibody which specifically binds to an epitope of a human ML-1 protein can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an antigen and an antibody that specifically binds to the antigen.

Typically, an antibody which specifically binds to a human ML-1 protein provides a detection signal at least 5-, 10-, or 20-fold higher than a detection signal provided with other proteins when used in an immunochemical assay. Preferably, antibodies which specifically bind to ML-1 proteins do not detect other proteins in immunochemical assays and can immunoprecipitations a human ML-1 protein from solution.

Antisense Oligonucleotides

Antisense oligonucleotides of the invention are complementary to a coding sequence for ML-1 protein. Preferably, an antisense oligonucleotide is at least 12 nucleotides in length, but can be between 12 and 330 nucleotides in length (e.g., 12, 15, 20, 25, 30, 35, 40, 45, 50, 75, 100, 125, 150, 175, 200, 225, 250, 275, 300, 325, or 330 nucleotides long).

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide. Phosphodiester and/or non-phosphodiester internucleotides linkages, such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters, can be used. See Brown, Meth. Mol. Biol. 20, 1-8, 1994; Sonveaux, Meth. Mol. Biol. 26, 1-72, 1994; Uhlmann et al., Chem. Rev. 90, 543-83, 1990.

Antisense oligonucleotides can be modified, for example, to affect stability, without affecting their ability to hybridize to a human ML-1 coding sequence. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-58, 1992; Uhlmann et al., Chem. Rev. 90, 543-84, 1990; Uhlmann et al., Tetrahedron. Lett. 215, 3539-42, 1987.

Ribozymes

Ribozymes can be used to inhibit ML-1 gene expression by cleaving an ML-1 RNA sequence, as is known in the art (e.g., Haseloff et al., U.S. Pat. No. 5,641,673). Methods of designing and constructing ribozymes have been developed and are well-known in the art (see Haseloff et al. Nature 334, 585-591, 1988).

Detecting ML-1 Expression Products

The invention provides methods of detecting cells that express a human ML-1 coding sequence. Cells in which expression of ML-1 can be detected include cells that normally express ML-1 (e.g., adult and fetal liver, lung, and ovary cells, and activated CD4+ T cells, basophils, peripheral blood monocytes, and mast cells), as well as host cells comprising ML-1 expression constructs. Either protein or RNA expression products can be detected. Methods that can be used to detect expression products of an ML-1 coding sequence include, but are not limited to, hybridization methods and assay techniques that include membrane, solution, or chip-based technologies for the detection and/or quantification of nucleic acid or protein.

For example, the presence of an mRNA encoding ML-1 protein can be detected by DNA-RNA hybridization or amplification, using single-stranded probes. Cells to be assayed for the presence of an RNA expression product can be present in a culture medium or can be tissue samples obtained from a research subject or patient (e.g., a bronchoalveolar lavage sample, a blood sample, or a tissue biopsy).

A variety of protocols for detecting and measuring the expression of a human ML-1 protein are known in the art. Many of these protocols use antibodies which specifically bind to human ML-1. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a human ML-1 protein can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216,1983). The ML-1 protein typically will be secreted into a culture medium; thus, the culture medium will normally be assayed for the presence of ML-1 protein.

Kits

Reagents of the invention, such as probes and antibodies, can be supplied in kits for use in detecting ML-1 expression products. Kits also may contain primers or sets of primers, which can be used to amplify ML-1-encoding sequences. Such kits may include instructions for using the reagents, buffers, reaction vessels, single or divided containers, and the like.

Increasing Expression of IL-6, IL-8, and ICAM-1

ML-1 protein can be used to increase expression of IL-6, IL-8, or ICAM-1 in an appropriate cell type, for example, to produce these proteins for therapeutic or research purposes. IL-6 and IL-8 are produced by many types of cells. For example, IL-6 is produced by monocytes, fibroblasts, endothelial cells, macrophages, T-cells, and B-lymphocytes, granulocytes, smooth muscle cells, eosinophils, chondrocytes, osteoblasts, mast cells, glial cells, and keratinocytes. IL-8 is produced by monocytes, lymphocytes, granulocytes, neutrophils, eosinophils, T cells, NK cells, fibroblasts, endothelial cells, bronchial epithelial cells, keratinocytes, hepatocytes, astrocytes, and chondrocytes. ICAM-1 is produced by a variety of human endothelial cells, such as airway epithelial cells, aortic endothelial cells, umbilical vein endothelial cells, and intestinal microvascular endothelial cells, and by many human cell lines, such as HeLa and WI38.

Any cell type capable of producing IL-6, IL-8, or ICAM-1 can be cultured in vitro using standard cell culture methods. ML-1 protein can be supplied to the cultured cells, for example by adding ML-1 protein to the cell medium or by providing the cultured cells with a polynucleotide delivery vehicle comprising ML-1 coding sequences which will be expressed in the cultured cells. Expression of IL-6, IL-8, or ICAM-1 is thereby increased relative to expression of IL-6, IL-8, or ICAM-1 in the absence of the ML-1 protein.

Screening Methods

The invention provides assays in which test compounds can be screened to identify candidate therapeutic agents that may be useful for treating airway inflammation, e.g., chronic or acute airway inflammation associated with asthma, chronic obstructive pulmonary disease (COPD), emphysema, an allergic inflammatory response, and cystic fibrosis. Candidates identified in screening assays can ultimately be tested for safety and efficacy in animal models or in humans. The identified candidates typically bind to and/or modulate either expression of the ML-1 gene, a biological activity of ML-1 protein, or the effective levels of ML-1 protein. For example, a candidate therapeutic agent may decrease expression of an ML-1 polynucleotide in a cell-free system or in a cell or tissue sample relative to expression in the absence of the candidate therapeutic agent. Alternatively, a candidate therapeutic agent may bind to human ML-1 protein, thereby preventing it from carrying out one or more of its biological functions (e.g., induction of IL-6, IL-8, or ICAM-1 expression in primary bronchial epithelial cells, induction of ERK1/2 activity in primary bronchial epithelial cells or in human umbilical vein endothelial cells, or increased neutrophil chemotaxis). Preferably, such candidate therapeutic agents affect the measured parameter by at least about 10, preferably about 50, more preferably about 75, 90, or 100% relative to the same parameter measured in the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by-chemical methods known in the art. If desired, test compounds can be obtained using any of the numerous combinatorial library methods known in the art including, but not limited to, biological libraries, spatially addressable parallel solid phase or solution phase libraries, synthetic library methods requiring deconvolution, the “one-bead one-compound” library method, and synthetic library methods using affinity chromatography selection.

Binding Assays

For binding assays, the test compound is preferably a small molecule that may bind to the ML-1 protein or polynucleotide such that normal biological activity is prevented. Examples of such small molecules include, but are not limited to, small inorganic molecules and peptides.

In binding assays, either the test compound or the ML-1 protein or polynucleotide can comprise a detectable label, such as a fluorescent, chemiluminescent, or radioactive moiety. A test compound that is bound to an ML-1 protein or polynucleotide can be detected, for example, by direct counting of radioemmission or by scintillation counting.

Alternatively, binding of a test compound to a human ML-1 protein or polynucleotide can be determined without labeling either of the interactants. For example, a microphysiometer (see McConnell et al., Science 257, 1906-1912, 1992) or a technique such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995) can be used to detect binding between two unlabeled interactants.

It may be desirable to immobilize either the ML,l protein or polynucleotide or the test compound to facilitate separation of bound from unbound forms of one or both of the interactants, as well as to accommodate automation of the assay. Thus, either the ML-1 protein or polynucleotide or the test compound can be bound to a solid support. Suitable solid supports include, but are not limited to, glass or plastic slides, tissue culture plates, microtiter wells, tubes, silicon chips, or particles such as beads (e.g., latex, polystyrene, or glass beads).

Any method known in the art can be used to attach the polypeptide, polynucleotide, or test compound to the solid support, including use of covalent and non-covalent linkages, passive absorption, or pairs of binding moieties attached respectively to the polypeptide, polynucleotide, or test compound and to the solid support. In one embodiment, the ML-1 protein is a fusion protein comprising a domain that allows the ML-1 protein to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed ML-1 protein. Avidin- and biotin-conjugated ML-1 proteins, polynucleotides, or test compounds can be bound to a solid support to which biotin or avidin, respectively, has been conjugated. Antibodies which specifically bind to ML-1 protein can be derivatized to a solid support. Test compounds can be bound to a solid support in an array, so that the location of individual test compounds can be tracked. Binding of a test compound to a human ML-1 protein or polynucleotide can be accomplished in any vessel suitable for containing the reactants. Examples of such vessels include microtiter plates, test tubes, and microcentrifuge tubes.

Test compounds and either ML-1 proteins or polynucleotides are incubated under conditions conducive to complex formation (e.g., physiological conditions for salt and pH). Following incubation, the solid support is washed to remove any unbound components. Binding of the interactants can be determined either directly or indirectly, as described above. Alternatively, the complexes can be dissociated from the solid support before binding is determined.

Methods for detecting such complexes, in addition to those described above for the immobilized complexes, include immunodetection of complexes using antibodies which specifically bind to the ML-1 protein or test compound and SDS gel electrophoresis under non-reducing conditions.

Screening for test compounds which bind to a human ML-1 polynucleotide also can be carried out in an intact cell. The ML-1 polynucleotide can be naturally occurring in the cell or can be introduced using techniques such as those described above. Binding of the test compound to the ML-1 polynucleotide is determined as described above.

In another embodiment, a human ML-1 protein is used as a “bait protein” in a two-hybrid assay or three-hybrid assay to identify other proteins which bind to or interact with ML-1 protein and modulate its activity. These techniques are well known and widely practiced in the art. See, e.g., U.S. Pat. No. 5,283,317; Zervos et al., Cell 72, 223-232, 1993; Madura et al, J. Biol. Chem. 268, 12046-12054, 1993; Bartel et al., BioTechniques 14, 920-924, 1993; Iwabuchi et al. Oncogene 8, 1693-1696, 1993.

Functional Activity

Test compounds can be tested for the ability to increase or decrease a functional activity of a human ML-1 protein. These functions include induction of IL-6, IL-8, or ICAM-1 in primary bronchial epithelial cells, as well as induction of ERK1/2 activity in primary bronchial epithelial cells or in human umbilical vein endothelial cells and increased neutrophil chemotaxis. Methods of measuring these activities are described in the Examples, below.

A test compound that decreases functional activity of a human ML-1 protein by at least about 10, preferably about 50, more preferably about 75, 90, or 100% is identified as a candidate therapeutic agent which may be useful for treating airway inflammation.

Gene Expression

In another embodiment, test compounds that increase or decrease ML-1 gene expression are identified. An ML-1 polynucleotide is contacted with a test compound, and the expression of an RNA or polypeptide product of the ML-1 polynucleotide is determined. The level of expression of appropriate mRNA or polypeptide in the presence of the test compound is compared to the level of expression of mRNA or polypeptide in the absence of the test compound. The test compound can then be identified as an inhibitor of ML-1 gene expression based on this comparison.

The level of ML-1 gene expression in a cell can be determined by methods well known in the art for detecting mRNA or polypeptide. Either qualitative or quantitative methods can be used. Levels of mRNA can be measured using Northern or dot blots, RNase protection assays, or other mRNA detection methods known in the art. The presence of polypeptide products of a human ML-1 polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a human ML-1 protein.

Screening can be carried out either in a cell-free assay system or a culture of intact cells. Any cell that expresses a human ML-1 polynucleotide can be used in a cell-based assay system. The ML-1 polynucleotide can be naturally occurring in the cell or can be introduced using techniques such as those described above. Either a primary culture or an established cell line, such as COS, HUVEC, or NHBE cells, can be used.

Therapeutic Methods

ICAM-1 expression is increased in airway diseases, such as bronchial asthma (13). In particular, a high level of ICAM-1 expression, along with inflammatory cell infiltration, has been demonstrated in bronchial biopsies from both stable asthmatics and subjects after allergen challenge (13, 20). Moreover, allergen challenge increases ICAM-1 expression in airway epithelium, correlating with eosinophil infiltration. Increased expression of ML-1 is also seen at sites of allergen challenge in patients with asthma Expression of ML-1 is detected in activated T cells and basophils, two important cell types involved in allergic responses. These observations suggest that ML-1 plays a role in the pathogenesis of airway inflammation by facilitating leukocyte recruitment and activation.

The invention thus provides methods of treating both acute and chronic airway inflammations, such as those associated with asthma, COPD, emphysema, allergic inflammatory responses, allergic rhinitis, and cystic fibrosis. Such methods can be used to reduce symptoms of the airway inflammation, such as bronchospasm, decreased air flow resistance, airway increased airway responsiveness to stimuli, brochoconstriction, cough, shortness of breath, mucus plugging, mucus hypersecretion, and mucosal edema. Neutrophil chemotaxis also may be inhibited.

These therapeutic methods of the invention involve administering to a patient an effective amount of a reagent that either decreases expression of human ML-1 gene or decreases effective levels of human ML-1 protein, thereby reducing symptoms of the airway inflammation relative to those in the absence of the reagent. Effective reagents include, but are not limited to, antibodies that specifically bind to ML-1 protein, antisense oligonucleotides, and ribozymes, as well as therapeutic agents identified using the screening assays described above.

Other therapeutic methods of the invention involve administering to a patient a reagent that increases expression of a human ML-1 gene or which increases effective levels of human ML-1 protein, such as a polynucleotide delivery vehicle encoding ML-1. Such methods are useful, for example, in cancer treatment, to induce or increase inflammation and thereby increase the effectiveness of a cancer vaccine.

Pharmaceutical Compositions

A reagent used in therapeutic methods of the invention is present in a pharmaceutical composition. Pharmaceutical compositions also comprise a pharmaceutically acceptable carrier, which meets industry standards for sterility, isotonicity, stability, and non-pyrogenicity and which is nontoxic to the recipient at the dosages and concentrations employed. The particular carrier used depends on the type and concentration of the therapeutic agent in the composition and the intended route of administration. If desired, a stabilizing compound can be included. Formulation of pharmaceutical compositions is well known and is described, for example, in U.S. Pat. Nos. 5,580,561 and 5,891,725.

Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. In some embodiments, pharmaceutical compositions are administered directly to the lung. The compositions can be administered to a patient alone or in combination with another therapeutic agent or agents. The other therapeutic agent(s) can be present in the same composition as the ML-1-specific reagent or can be administered in a separate composition, either concurrently or simultaneously.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

In the examples below, the statistical significance of differences was determined by analysis of variance (ANOVA). In Examples 1-5, any difference with p value less than 0.01 was considered significant. In Examples 6-9, any difference with p values less than 0.05 was considered significant. When ANOVA indicated a significant difference, the Scheffe F-test was used to determine the difference between groups.

### EXAMPLE 1

Full-Length cDNA Sequence of ML-1 Gene

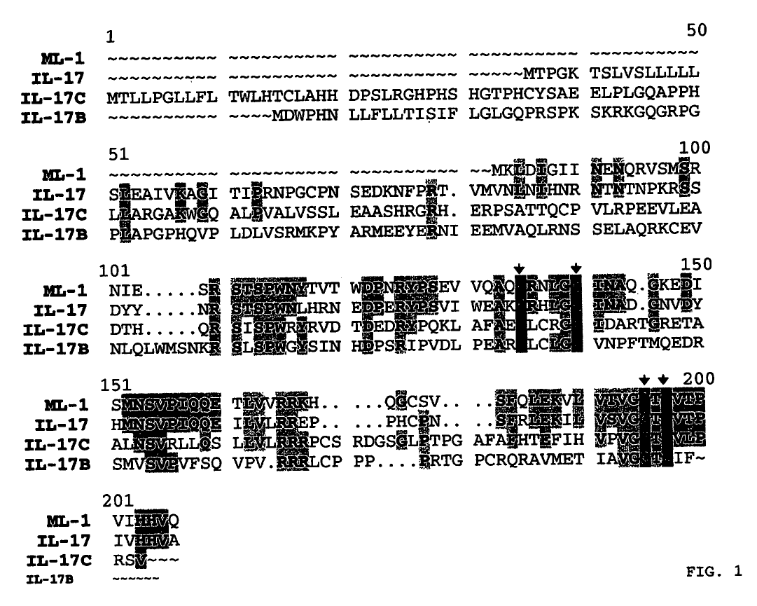

As part of a positional cloning study of a region on chromosome 6p for susceptibility gene discovery for an inherited disease, a potential coding-region sequence with homology to human IL-17 was identified from a genomic DNA clone, PAC108C2* (Sanger Centre Database, URL address: http file type, www. host server, domain name sanger.ac.uk), using the GenScan prediction program. The predicted expressed sequence with a centromeric-telomeric orientation was composed of 2 exons of 221 and 238 basepair, respectively. RT-PCR and sequencing analysis of activated, human allergen-specific T-cell clones confirmed the predicted sequence and the splicing sites between the exons. However, the open-reading frame utilized a start codon 129 basepairs 3′ to the predicted start site, encompassing a 92-bp segment in the first exon. A 9476 full-length cDNA was obtained using both 5′- and 3′-RACE, revealing a transcription start site 346 basepairs upstream of the start codon and a poly(A) sequence 271 basepairs 3′ to the stop codon. An alignment of the predicted amino acid sequence of ML-1 with the known sequences of IL-17 and the other members of the IL-17 family shows that, while there is 70% amino acid sequence homology between ML-1 and IL-17, there is only 20% amino acid identity between ML-1 and the three other family members (FIG. 1). The alignment shows several conserved amino acids, including a tryptophan residue and four cysteines in the C-terminal halves of the proteins.

To obtain a full-length cDNA sequence, both 5′- and 3′-RACE were performed using cDNAs from ragweed allergen-activated PBMCs as templates. For 3′-RACE, cDNAs were amplified using poly(dT) and a predicted exon sequence primer, 5′-GGCATCATCAATGAAAACCAG-3′ (SEQ ID NO:6). The PCR products were run on a 1% low-melting agarose gel, purified using a GeneClean kit (Qbiogene, Carlsbad, Calif.), and subjected to a nested PCR using an internal sequence primer, 5′-TTCCATGTCACGTAACATCG-3′ (SEQ ID NO:7). The products were then cloned and sequenced. For 5′-RACE, cDNAs were first tailed with poly(da) oligonucleotides using TdT enzyme, purified using Sephadex G25 spin columns, and subjected to nested PCR reactions using poly(dT), a coding-region sequence (5′-TCACCAGCACCTTCTCCAAC-3′; SEQ ID NO:8), and an internal sequence (5′-AAGAAACAGAGCAGCCTTGG-3′; SEQ ID NO:9) primer. The PCR products were then cloned and sequenced.

### EXAMPLE 2

Cells, Isolation of RNAs, and Expression Analysis; Tissue and Cellular Distribution of the ML-1 Gene

Tissue distribution data for IL-17 and ML-1 were acquired using Rapid-Scan gene expression panels for human tissues (OriGene Technologies, Inc., Rockville, Md.) according to the manufacturer's instructions, with 5 mM magnesium and ML-1-specific primer pairs. The sequences of primers for ML-1 were as follows: forward, 5′-GGCATCATCAATGAAAACCAG-3′ (SEQ ID NO:10) and reverse, 5′-TCACCAGCACCTTCTCCAAC-3′ (SEQ ID NO:11). PCR products were visualized on an ethidium bromide-containing gel and photographed. Tissues were graded on a 0-4 grading system, based on visualization of bands at the concentrations of cDNA provided by the manufacturer (grade 4=product obtained with >1 pg/ml; grade 3=product obtained with >10 pg/ml; grade 2=product obtained with >100 pg/ml; grade 1=product obtained with 1000 pg/ml; grade 0=no product obtained with 1000 pg/ml). Appropriate normalization of cDNA provided by the manufacturer was confirmed by PCR amplification for the constitutive marker gene, β actin.

Peripheral blood monocytes (PBMCs) were isolated from the blood of allergic subjects. Human allergen-specific T-cell clones were generated by limiting dilution cloning and sub-cloning from two atopic subjects, followed by biweekly stimulation of T cells with ragweed allergen extract or purified Amb a 1 (a major ragweed allergen) together with irradiated, autologous PBMCs as antigen presenting cells, as described previously (17). The cytokine profiles of T-cell clones were determined as described in (17). Basophils were isolated and purified to homogeneity (>98% purity) following double Percoll density centrifugation and negative selection using a cocktail of monoclonal antibodies (CD2, CD3, CD14, CD16, CD24, CD34, CD36, CD45RA, CD56, glycophorin) and magnetic colloid beads (Stemcell Technologies, Inc., Vancouver, Canada) (see 18). Basophils were activated by stimulation with anti-IgE antibodies as previously described (18).

ML-1 gene expression was also assayed from bronchoalveolar lavage (BAL) cells of four asthmatic patients challenged with either allergen (ragweed, 100 PNU) or with a saline control, as described in (19). The BAL cells were collected 19 hours after challenge. Total RNA was isolated from ragweed-activated PBMCs (5×10⁶ cells; 6 hours after stimulation), cloned T cells (2×10⁶ cells; 6 hours after ragweed allergen stimulation), and basophils (2×10⁶ cells; 4 hours after stimulation) using RNAzolB according to the manufacturer's instructions (TelTest, Friendswood, Tex.). Complementary DNAs were synthesized from 500 ng of total RNA in the presence of MMLV reverse transcriptase (1 U/reaction; Sigma, St. Luis, Mo.), oligo(dT) primer, and reaction buffer at 42° C. for 90 minutes, followed by PCR. Each cDNA sample was amplified (30 cycles of 1 minute at 95° C., 1 minute at 60° C., and 1 minute at 72° C.) using a pair of ML-1 sequence-specific primers (see above). The sequences of primers for the housekeeping gene, G3PDH, were as follows: forward, 5′-ACCACAGTCCATGCCATCAC-3′ (SEQ ID NO:12); reverse, 5′-TCCACCACCCTGTTGCTGTA-3′ (SEQ ID NO:13). The expected sizes for the ML-1 and G3PDH PCR products were 268 basepairs and 450 basepairs, respectively.

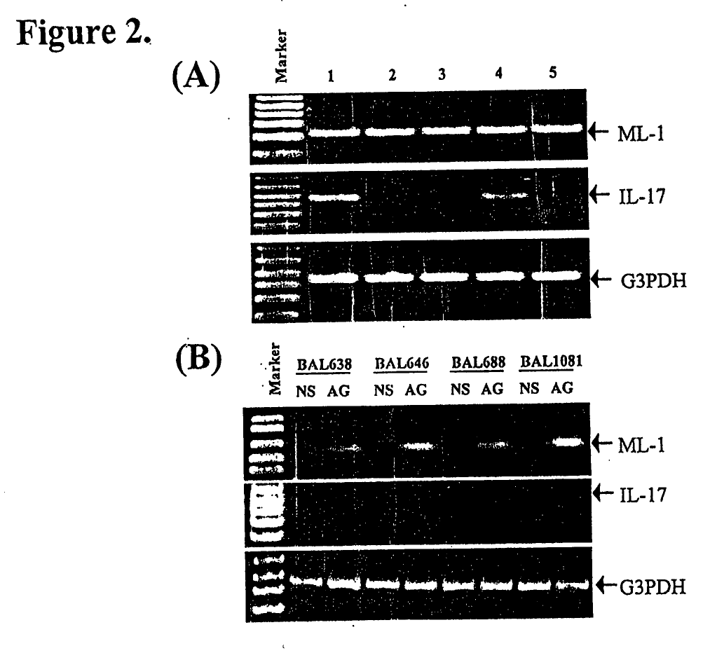

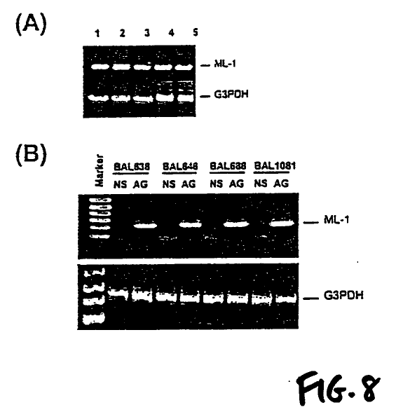

The expression of ML-1 in various human tissues was examined using PCR and the Rapid-Scan gene expression panels for human tissues. ML-1 was strongly expressed in liver, lung, spleen, placenta, adrenal gland, ovary, and fetal liver, as shown in Table 1. Interestingly, IL-17 expression was not detected in liver, lung, ovary, or fetal liver. In addition, ML-1 expression was clearly evident in five different cell types after activation: ragweed allergen-stimulated PBMCs, ragweed allergen-specific T-cell clones with Th0 (clone 12), Th1 (clone 2B7), and Th2 (clone 2D2) phenotypes, and activated basophils (FIG. 2A). Interestingly, low levels of gene expression for ML-1 were detected in resting Th1 cells, but not in any other resting immune cells. See FIG. 8.

To determine the in vivo relevance of ML-1 gene expression, we performed analyses of gene expression in BAL cells from asthmatic subjects challenged with allergen or saline control. FIG. 2B shows representative data demonstrating that, while no detectable expression of ML-1 was seen in BALs from saline-challenged sites, ML-1 gene expression was clearly demonstrated in BALs from allergen-challenged sites of all four study subjects.

TABLE 1

| Tissue | IL-17 | ML-1 |
|---|---|---|
| Spleen | + | ++ |
| Liver | 0 | +++ |
| Lung | 0 | +++ |
| Small Intestine | + | 0 |
| Stomach | + | + |
| Testis | ++ | + |
| Placenta | 0 | ++ |
| Adrenal Gland | ++ | ++ |
| Ovary | 0 | ++ |
| Uterus | 0 | + |
| Prostate | + | + |
| Skin | 0 | + |
| Fetal Liver | 0 | ++ |

Grade 0-Brain; Heart; Kidney; Colon; Muscle; Salivary Gland; Thyroid Gland; Pancreas; Bone Marrow; Fetal Brain.

### EXAMPLE 3

Recombinant ML-1-His fusion Proteins

The coding sequence of ML-1 was amplified by PCR and subcloned into the Bam HI and Sal I sites of pcDNA 3.1 (Invitrogen, Carlsbad, Calif.) to generate a C-terminal fusion gene with the His and cMyc tags. The vector pcDNA 3.1 was transfected into COS-7 cells by an Effectene Reagent (Qiagen, Chatsworth, Calif.) according to the manufacturer's instructions. Two days after transfection, the supernatants were concentrated over Centricon-10 columns (Amicon, Beverly, Mass.) and subjected to affinity purification by Ni—NTA agarose beads (Qiagen, Chatsworth, Calif.) for His-tagged proteins. To examine the protein expression, SDS-PAGE analysis was performed on the affinity-purified, recombinant proteins under a reducing condition, followed by Western Blot analysis using anti-His monoclonal antibody (Santa Cruz Biotechnology, Inc., Santa Cruz, Calif.).

### EXAMPLE 4

Analysis of IL-8 and ICAM-Expression

Primary human bronchial epithelial (NHBE) cells were purchased from Clonetics (San Diego, Calif.) and cultured according to the manufacturer's instructions. The cells were treated with IL-17 (100 ng/ml), ML-1 (100 ng/ml), or a control His-tagged protein (Positope, 100 ng/ml; Invitrogen, Carlsbad, Calif.). The affinity-purified control His protein was dissolved in the same buffer as ML-1. Total RNA was extracted using Rneasy (Qiagen, Chatsworth, Calif.) from 1×10⁶ cells 4 hours after stimulation or exchange of media. The protocol for cDNA synthesis was that described above. For PCR, the sequences of PCR primers were based on the human IL-8 cDNA sequence. The sequences of PCR primers for IL-8 were: forward, 5′-TCTGCAGCTCTGTGTGAAG-3′ (SEQ ID NO:14) and reverse, 5′-TAATTTCTGTGTTGGCGCA-3′ (SEQ ID NO:15). The amplification reaction was performed for 23 cycles with denaturation at 94° C. for 45 seconds, annealing at 56° C. for 45 seconds, and extension at 72° C. for 45 seconds PCR products were detected by ethidium bromide staining. The expected size for IL-8 was 154 basepairs. IL-8 protein levels in the collected supernatants were determined with a commercially available ELISA kit (Biosource, Camarillo, Calif.) according to the manufacturer's instructions.

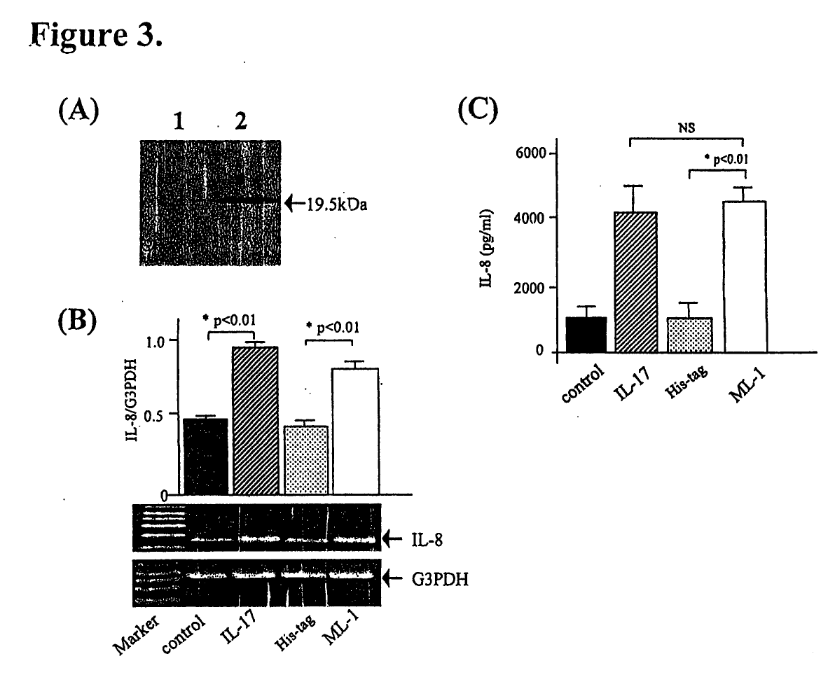

To assess the biological functions of ML-1 in comparison to IL-17, His-tagged rML-1 protein was expressed in COS-7 cells and affinity purified (FIG. 3A). Primary bronchial epithelial cells (NHBE) were treated with either affinity purified ML-1 or a His-tagged control protein and assayed for IL-8 and ICAM-1 expression. Similar to IL-17, ML-1 enhanced IL-8 transcript and protein expression at 48 hours in NHBE cells (FIGS. 3B and 3C), suggesting that ML-1 is involved in neutrophil recruitment into the airway. It has been previously shown that IL-17 alone is not able to induce ICAM-1 expression on airway epithelial cells (12), but acts synergistically with IFN-γ to induce of ICAM-1 expression on epithelial cells. Increased of ICAM-1 expression is associated with airway inflammation seen in bronchial asthma (13).

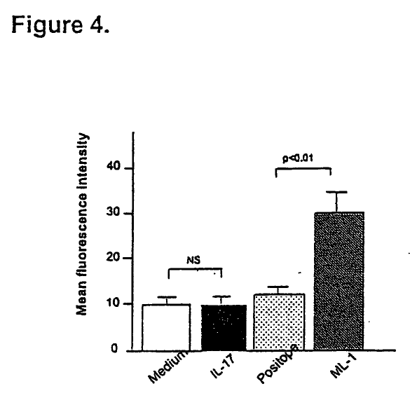

To determine whether ML-1 is able to induce ICAM-1 expression on NHBE cells, surface expression of ICAM-1 on NHBE cells was measured and the effects of ML-1 and IL-17 were compared. FIG. 4 demonstrates that ICAM-1 was constitutively, but weakly, expressed on NHBE cells (MFI=12.26±1.77). IL-17 (100 ng/ml) alone did not affect ICAM-1 surface expression at 48 hours (MFI=10.07±1.22). In contrast, ML-1 (100 ng/ml) significantly induced ICAM-1 expression at 48 hours (MFI=31.42±4.39) (p<0.01) when compared to expression seen in cells stimulated with IL-17 and a His-tagged protein (MFI=12.99±2.09).

Thus, the biological effects of ML-1 and IL-17 are different. While both IL-17 and ML-1 induce IL-8 expression from NHBE cells, IL-17 does not induce ICAM-1 expression in human bronchial epithelial cells. By comparison, ML-1 markedly induces ICAM-1 expression. These different biological effects of ML-1 and IL-17 suggest that these two cytokines may signal via different cell surface receptors.

### EXAMPLE 5

### Analysis of ICAM-1 Surface Protein Expression by Flow Cytometry

NHBE cells were treated with IL-17 (100 ng/ml), ML-1 (100 ng/ml), or a control His protein (Positope, 100 ng/ml). The cells were harvested following treatment with 0.1% trypsin-0.02% EDTA at 37° C. for 6 minutes and suspended in PBS containing 2% BSA and 0.02 % sodium azide. One million cells were incubated with a mouse anti-human ICAM-1 monoclonal antibody (R&D Systems, Minneapolis, Minn.) on ice for 30 minutes. After three washes in PBS, the cells were incubated with fluorescein isothiocyanate-conjugated goat anti-mouse IgG (Bio Rad, Hercules, Calif.) on ice for 30 minutes. After three additional washes in PBS, the cells were resuspended in PBS and immediately analyzed with a FACScan flow cytometer (Becton Dickinson, Mountain View, Calif.). In control samples, staining was performed using isotype-matched control antibodies. Indices are expressed as mean±SD (n=4).

### EXAMPLE 6

Generation of Recombinant Human ML-1 Protein

Human recombinant ML-1 was generated as described in (27). The coding sequence of ML-1 was amplified by PCR and subcloned into pcDNA 3.1 (Invitrogen, Carlsbad, Calif.) to generate a C-terminal His fusion gene. The vector pcDNA 3.1 was transfected into COS-7 cells by an Effectene Reagent (Qiagen, Chatsworth, Calif.) according to the manufacturer's instructions. ML-1 was purified with affinity purification by Ni—NTA agarose beads (Qiagen) for His-tagged proteins. The concentration of ML-1 protein was quantified by Bradford assay (BIO-RAD, Hercules, Calif.), and the protein was stored at −80° C. until used.

### EXAMPLE 7

Cell Cultures

Primary bronchial epithelial cells (PBECs) were purchased from Clonetics (San Diego, Calif.) and cultured in bronchial epithelial basal medium (Clonetics) containing 0.5 ng/ml human recombinant epidermal growth factor (EGF), 52 mg/ml bovine pituitary extract, 0.1 mg/ml retinoic acid, 0.5 mg/ml hydrocortisone, 5 mg/ml insulin, 10 mg/ml transferrin, 0.5 mg/ml epinephrine, 6.5 ng/ml triiodothyronine, 50 mg/ml gentamicin, and 50 pg/ml amphotericin-B (Clonetics).

Human umbilical vein endothelial cells (HUVECs) were obtained from Clonetics and cultured in endothelial cell growth media (EGM; Clonetics) containing 12 μg/ml bovine brain extract, 10 ng/ml EGF, 1 μg/ml hydrocortisone, 50 μg/ml gentimicin, 50 ng/ml amphotericin B, and 5% FBS amphotericin-B (Clonetics).

Both PBECs and HUVECs were incubated at 37° C. in humidified 5% CO₂ and cultured for no more than three passages prior to analysis.

### EXAMPLE 8

Analysis of IL-6 and IL-8 in PBECs and HUVECs

PBECs and HUVECs were treated with ML-1 100 ng/ml of ML-1 for various time points. Total RNA was extracted using RNeasy (Qiagen) from 1×10⁶ cells 4 hours after stimulation or exchange of media. The protocol for cDNA synthesis was that described above. For PCR, the sequences of PCR primers were based on the human IL-8 cDNA sequence. The sequences of PCR primers for IL-8 were: forward, 5′-TCTGCAGCTCTGTGTGAAG-3′ (SEQ ID NO:14) and reverse, 5′-TAATTTCTGTGTTGGCGCA-3′ (SEQ ID NO:15). For IL-6, the primers were: forward, 5′-ATGAACTCCTTCTCCACAAGCGC-3′ (SEQ ID NO:16) and reverse, 5′-GAAGAGCCCTCAGGCTGGACTG-3′ (SEQ ID NO: 17).

The amplification reaction was performed for 23 cycles with denaturation at 94° C. for 45 seconds, annealing at 56° C. for 45 seconds, and extension at 72° C. for 45 seconds. PCR products were detected by ethidium bromide staining and normalized by the intensity of an amplified housekeeping gene, G3PDH (see above). The expected sizes for IL-8 and IL-6 were 154 basepairs and 628 basepairs, respectively. IL-6 and IL-8 protein levels in the collected supernatants were determined with a commercially available ELISA kit (Biosource, Camarillo, Calif.) according to the manufacturer's instructions.

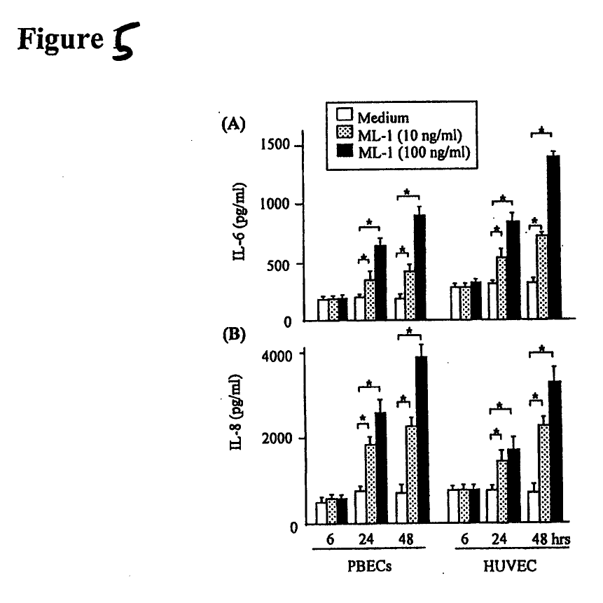

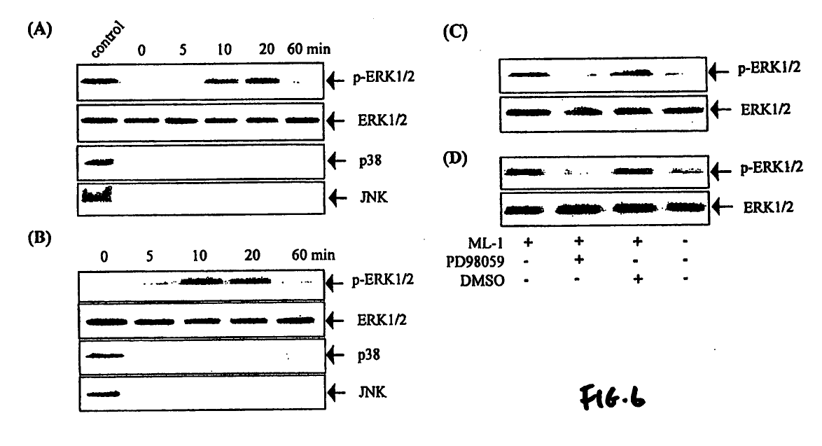

Stimulation of PBECs and HUVECs with ML-1 elicited a time-dependent increase in IL-6 and IL-8 production (FIGS. 5A and 5B). ML-1 significantly induced IL-6 and I-8 production at two different doses (10 and 100 ng/ml) and at two different time points (24 and 48 hours). To determine the signaling pathway involved in ML-1-induced cytokine expression, untreated or ML-1-stimulated cells were lysed at various time points and Western blotting analyses were performed using various antibodies against members of the MAP kinase family, (ERK1/2, p38, and JNK). No activation of p38 and JNK kinases was seen at any time points (FIG. 6A). Western blotting analysis revealed the phosphorylation of ERK1/2 which reached a maximum at 20 and 10 minutes in PBECs and HUVECs, respectively, and which returned to baseline levels by 60 minutes (FIGS. 6A and 6B). In addition, pre-incubation of the cells with 10 μM of the a MEK inhibitor, PD98059, diminished the activation of ERK1/2 in both PBECs and HUVECs. Pre-incubation of DMSO did not affect the phosphorylation of ERK1/2 (FIGS. 6C and 6D).

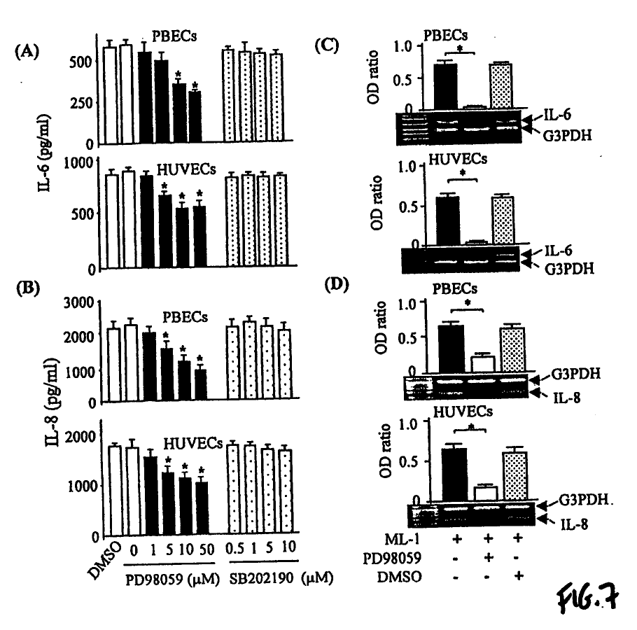

To determine whether the activation of ERK1/2 is necessary for the stimulation of IL-6 and IL-8 production in both cell types, the cells were stimulated with ML-1 in the presence or the absence of PD98059. As demonstrated in FIG. 7, PD98059 inhibited the production of both IL-6 and IL-8 in a dose-dependent manner, while pretreatment of the cells with DMSO did not affect cytokine protein release in either PBECs or HUVECs (FIGS. 7A and 7B). Decreased levels of IL-6 and IL-8 gene expression were also noted in PD98059-treated cells, but not in vehicle-treated cells, suggesting that the inhibitory effect was at the level of transcription (FIGS. 7C and 7D).

### EXAMPLE 9

Detection of MP Kinases

For analysis of activation of MAP kinases, PBECs were treated with IL-17 (100 ng/ml) for various time periods, with or without the MEK 1/2 inhibitor PD98059 (1-50 mM; Calbiochem, La Jolla, Calif.) (15), the p38 inhibitor SB202190 (0.5-10 μM; ref. 16), or a vehicle control, DMSO (Me₂SO) for 1 hour. The final concentration of DMSO did not exceed 0.1% (v/v). Following the treatment, the cells were washed with ice-cold PBS. Cell pellets were immediately lysed in cold lysis buffer [20 mM Tris (pH 7.4), 4 mM EDTA, 2 mM EGTA, 1 mM PMSF, 100 mg/ml aprotinin, 200 mg/ml leupeptin, 50 mM NaF, 5 mM NaRP₂O₇, 1 mM Na₃VO₄, and 1% Nonidet P40; all purchased from Sigma]. Extracts (1×10 6 cell equivalents/lane) were suspended with an equal volume of 2× loading buffer [0.1 M Tris-HCl (pH 6.8)], 4% SDS, 0.005% bromophenol blue, and 20% glycerol) containing 2-ME (0.7 M) and subjected to 4-20% Tris-glycine gel electrophoresis (NOVEX, San Diego, Calif.). Gels were then transferred to PVDF membranes (BIO-RAD, Hercules, Calif.) with a Trans Blot apparatus (NOVEX). The membranes were immersed overnight in This-buffered saline/Tween 20 containing 5% nonfat dry skim milk (Carnation, Los Angeles, Calif.).

Immunoreactive proteins were detected using antibodies against various kinases and phosphorylated kinases. The antibodies used were: rabbit anti-ERK1/2 antibody, anti-phospho-ERK1/2 antibody, anti-p38 antibody, anti-JNK antibody, and anti-phospho-JNK antibody (New England Biolabs, Beverly, Mass.), and rabbit anti-phospho-p38 antibody (Santa Cruz Biotechnology, Santa Cruz, Calif.). All antibodies were suspended in Tris-buffered saline/Tween 20 containing 5% skim milk for 1 hour. After washing, the membranes were incubated with peroxidase-linked donkey anti-rabbit Ig antibody (Amersham, Arlington Heights, Ill.) for 1 hour. After washing, membrane-bound anti-rabbit Ig antibody was visualized with enhanced chemiluminescence. Western blotting detection reagents (Amersham) and Hyper ECL luminescence detection film (Amersham) were used. In some cases, protein standards (New England Biolabs) were run in parallel and served as positive controls.

### REFERENCES

1. Rouvier et al., 1993. CTLA-8, cloned from an activated T cell, bearing AU-rich messenger RNA instability sequences, and homologous to a herpesvirus saimiri gene. J. Immunol. 150:5445.
2. Yao et al., 1995. Human IL-17: a novel cytokine derived from T cells. J. Immunol. 155:5483.
3. Fossiez et al. 1996. T cell interleukin-17 induces stromal cells to produce proinflammatory and hematopoietic cytokines. J. Ex. Med. 183:603.
4. Albanesi et al. 2000. Interleukin-17 is produced by both Th1 and Th2 lymphocytes, and modulates interferon-gamma- and interleukin-4-induced activation of human keratinocytes. J Invest Dermatol 115:81.
5. Cai et al. 1998. Regulation of granulocyte colony-stimulating factor gene expression by interleukin-17. Immunol. Lett. 62:51.
6. Attur et al. 1997. Interleukin-17 up-regulation of nitric oxide production in human osteoarthritis cartilage. Arthritis Rheum 40:1050.
7. Van Kooten et al. 1998. Interleukin-17 activates human renal epithelial cells in vitro and is expressed during renal allograft rejection. J. Am. Soc. Nephrol. 9:1526.
8. Matusevicius et al. 1999. Interleukin-17 mRNA expression in blood and CSF mononuclear cells is augmented in multiple sclerosis. Mult Scler. 2:101.
9. Ziolkowska et al. 2000. High levels of IL-17 in rheumatoid arthritis patients: IL-15 triggers in vitro IL-17 production via cyclosporin A-sensitive mechanism. J. Immunol. 164:2832.
10. Wong et al. 2000. Elevation of proinflammatory cytokine (IL-18, IL-17, IL-12) and Th2 cytokine (IL-4) concentrations in patients with systemic lupus erythematosus. Lupus 9:589.
11. Laan et al. 1999. Neutrophil recruitment by human IL-17 via C—X—C chemokine release in the airways. J. Immunol. 162:2347.
12. Kawaguchi et al. 1999. Effect of IL-17 on ICAM-1 expression of human bronchial epithelial cells, NCI—H 292. arerugi 1184.
13. Manolitsas et al. 1994. The expression of intercellular adhesion molecule-1 and the beta 1-integrins in asthma. Eur. Respir. J. 7:1439.
14. Li et al. 2000. Cloning and characterization of IL-17B and IL-17C, two new members of the IL-17 cytokine family. Proc. Natl. Acad. Sci. USA 97:773.
15. Shi et al. 2000. A novel cytokine receptor-ligand pair. Identification, molecular characterization, and in vivo immunomodulatory activity. J. Biol. Chem. 275:19167.
16. Lee et al. 2000. IL-17E, a novel proinflammatory ligand for the IL-17 receptor homolog IL-17Rh1. J. Biol. Chem. 276,1660-64.
17. Essayan et al. Clonal diversity of interleukin-4 and interleukin-13 expression in human allergen-specific T lymphocytes. 1996. Clonal diversity of IL-4 and IL-13 expression in human allergen-specific T lymphocytes. J. Allergy Clin. Immunol. 98:1035.
18. Schroeder et al., 1994. IgE-dependent IL-4 secretion by human basophils. The relationship between cytokine production and histamine release in mixed leukocyte cultures. J. Immunol. 153:1808.
19. Huang et al. 1995. Interleukin-13 expression at the sites of allergen challenge of patients with asthma J. Immunol. 155:2688.
20. Bentley et al. 1993. Expression of endothelial and leukocyte adhesion molecules interacellular adhesion molecule-1, E-selectin, and vascular cell adhesion molecule-1 in the bronchial mucosa in steady-state and allergen-induced asthma. J Allergy Clin. Immunol. 92:857
21. Kwon et al. 1994. Am. J. Physiol. 267:L398-405.
22. Chen & Manning. 1996. Cytokine. 8:58-65.
23. Steerenberg et al., 1998. Exp. Lung Res. 24:85-100.
24. Matsukura et al., 1996. J. Allergy Clin. Immunol. 98:1080-1087.
25. Li et al., 2000. Proc. Natl. Acad. Sci. USA. 97:773-778.
26. Shi et al., 2000. J. Biol. Chem. 275:19167-19176.
27. Kawaguchi et al., 2001. J. Immunol. 67:4430-4435.
28. Reibman et al., 2000. J. Immunol. 165:1618-1625.
29. Chayama et al., 2001. Proc. Natl. Acad. Sci. USA. 2001;98:4599-4604.
30. Kampen et al., 2000. Blood. 95:1911-1917.
31. Surapisitchat et al., 2001. Proc. Natl. Acad. Sci. USA. 98:6476-6481.
32. Alessi et al., 1995. J. Biol. Chem. 270:27489-27494.
33. Laan et al., 2001. Br. J. Pharmacol. 133:200-206.
34. Kawaguchi et al., 2001. J. Allergy Clin. Immunol. 108:804-809.
35. Hashimoto et al., 2000. Clin. Exp. Allergy. 30:48-55.
36. Hashimoto et al., 2000. Am. J. Respir. Crit. Care. Med. 161:280-285.
37. Griego et al., 2000. J. Immunol. 165:5211-5220.
38. Kujime et al., 2000. J. Immunol. 164:3222-3228.
39. May et al., 1998. Am. J. Physiol. 274:C789-798.
40. Goebeler et al., 1999. Blood. 93:857-865.

1. an isolated and purified human ML-1 protein comprising a first polypeptide segment comprising the amino acid sequence shown in SEQ ID NO:2.

2. The protein of claim 1 further comprising a second polypeptide segment comprising an amino acid sequence which is not a human ML-1 amino acid sequence, wherein the second polypeptide segment is joined to the first polypeptide segment by means of a peptide bond.

3. An isolated and purified human ML-1 protein comprising an amino acid sequence which differs from the amino acid sequence shown in SEQ ID NO:2 by between one and ten conservative amino acid substitutions and which (a) induces expression of IL-6, IL-8, and ICAM-1 in primary bronchial epithelial cells, (b) induces ERK1/2 activity in human primary bronchial epithelial cells and in human umbilical vein endothelial cells, (c) increases neutrophil chemotaxis, and (d) is expressed in activated CD4+ T cells, basophils, peripheral blood monocytes, and mast cells.

4-10. (canceled)

11. An isolated and purified polynucleotide which encodes a human ML-1 protein comprising the amino acid sequence shown in SEQ ID NO:2.

12. The polynucleotide of claim 11 which comprises the nucleotide sequence shown in SEQ ID NO:1.

13. The isolated and purified polynucleotide of claim 11 which is a cDNA molecule.

14. The cDNA molecule of claim 13 which comprises the nucleotide sequence shown in SEQ ID NO:1.

15-21. (canceled)

22. The isolated and purified polynucleotide of claim 11 which is in an expression construct.

23-71. (canceled)


SEQUENCE LISTING (17 sequences)

SEQ ID NO: 1 - DNA, 330 bp, Homo sapiens
atgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaa

SEQ ID NO: 2 - protein, 109 aa, Homo sapiens
MKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 3 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 4 - protein, 196 aa, Homo sapiens
MTLLPGLLFLTWLHTCLAHHDPSLRGHPHSHGTPHCYSAEELPLGQAPPHLLARGAKWGQALPVALVSSLEAASHRGRHRPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 5 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 6 - DNA, 21 bp, Homo sapiens
ggcatcatcaatgaaaaccag

SEQ ID NO: 7 - DNA, 20 bp, Homo sapiens
ttccatgtcacgtaacatcg

SEQ ID NO: 8 - DNA, 20 bp, Homo sapiens
tcaccagcaccttctccaac

SEQ ID NO: 9 - DNA, 20 bp, Homo sapiens
aagaaacagagcagccttgg

SEQ ID NO: 10 - DNA, 21 bp, Homo sapiens
ggcatcatcaatgaaaaccag

SEQ ID NO: 11 - DNA, 20 bp, Homo sapiens
tcaccagcaccttctccaac

SEQ ID NO: 12 - DNA, 20 bp, Homo sapiens
accacagtccatgccatcac

SEQ ID NO: 13 - DNA, 20 bp, Homo sapiens
tccaccaccctgttgctgta

SEQ ID NO: 14 - DNA, 19 bp, Homo sapiens
tctgcagctctgtgtgaag

SEQ ID NO: 15 - DNA, 19 bp, Homo sapiens
taatttctgtgttggcgca

SEQ ID NO: 16 - DNA, 23 bp, Homo sapiens
atgaactccttctccacaagcgc

SEQ ID NO: 17 - DNA, 22 bp, Homo sapiens
gaagagccctcaggctggactg